(19) United States
(12) Patent Application Publication
(10) Pub. No.: US 2012/0265279 A1
(43) Pub. Date: Oct. 18, 2012

(54) SENSING DEVICE FOR INDICATING POSTURE OF PATIENT IMPLANTED WITH A NEUROSTIMULATION DEVICE

(75) Inventor: Changfang Zhu, Valencia, CA (US)

(73) Assignee: BOSTON SCIENTIFIC NEUROMODULATION CORPORATION, Valencia, CA (US)

(21) Appl. No.: 13/446,191

(22) Filed: Apr. 13, 2012

Related U.S. Application Data
(60) Provisional application No. 61/474,977, filed on Apr. 13, 2011.

(51) Int. Cl. A61N 1/05

(52) U.S. Cl. 607/116

(57) ABSTRACT

An implantable medical device comprises a medical component configured for performing a medical function in a patient, an orientation sensitive component including a housing having a cavity, a movable object configured for being displaced within the cavity in response to the change in the direction of a force applied to the movable object, and a plurality of fixed sensors spaced apart within the cavity for sensing a location of the movable object within the cavity, and monitoring circuitry configured for determining the orientation of the implantable medical device based on the sensed location of the movable object.

### RELATED APPLICATION DATA

The present application claims the benefit under 35 U.S.C. §119 to U.S. provisional patent application Ser. No. 61/474,977, filed Apr. 13, 2011. The foregoing application is hereby incorporated by reference into the present application in its entirety.

### FIELD OF THE INVENTION

The present invention relates to tissue stimulation systems, and more particularly, to a system and method for indicating the posture of a patient for improved neurostimulation.

### BACKGROUND OF THE INVENTION

Implantable neurostimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) systems have long been accepted as a therapeutic modality for the treatment of chronic pain syndromes, and the application of tissue stimulation has begun to expand to additional applications such as angina pectoralis and incontinence. Deep Brain Stimulation (DBS) has also been applied therapeutically for well over a decade for the treatment of refractory chronic pain syndromes, and DBS has also recently been applied in additional areas such as movement disorders and epilepsy. Further, in recent investigations, Peripheral Nerve Stimulation (PNS) systems have demonstrated efficacy in the treatment of chronic pain syndromes and incontinence, and a number of additional applications are currently under investigation. Furthermore, Functional Electrical Stimulation (FES) systems, such as the Freehand system by NeuroControl (Cleveland, Ohio), have been applied to restore some functionality to paralyzed extremities in spinal cord injury patients.

These implantable neurostimulation systems typically include one or more electrode carrying stimulation leads, which are implanted at the desired stimulation site, and a neurostimulator (e.g., an implantable pulse generator (IPG)) implanted remotely from the stimulation site, but coupled either directly to the stimulation lead(s) or indirectly to the stimulation lead(s) via a lead extension. The neurostimulation system may further comprise an external control device to remotely instruct the neurostimulator to generate electrical stimulation pulses in accordance with selected stimulation parameters.

Electrical stimulation energy may be delivered from the neurostimulator to the electrodes in the form of an electrical pulsed waveform. Thus, stimulation energy may be controllably delivered to the electrodes to stimulate neural tissue. The combination of electrodes used to deliver electrical pulses to the targeted tissue constitutes an electrode combination, with the electrodes capable of being selectively programmed to act as anodes (positive), cathodes (negative), or left off (zero). In other words, an electrode combination represents the polarity being positive, negative, or zero. Other parameters that may be controlled or varied include the amplitude, width, and rate of the electrical pulses provided through the electrode array. Each electrode combination, along with the electrical pulse parameters, can be referred to as a “stimulation parameter set.”

With some neurostimulation systems, and in particular, those with independently controlled current or voltage sources, the distribution of the current to the electrodes (including the case of the neurostimulator, which may act as an electrode) may be varied such that the current is supplied via numerous different electrode configurations. In different configurations, the electrodes may provide current or voltage in different relative percentages of positive and negative current or voltage to create different electrical current distributions (i.e., fractionalized electrode combinations).

As briefly discussed above, an external control device can be used to instruct the neurostimulator to generate electrical stimulation pulses in accordance with the selected stimulation parameters. Typically, the stimulation parameters programmed into the neurostimulator can be adjusted by manipulating controls on the external control device to modify the electrical stimulation provided by the neurostimulator system to the patient. Thus, in accordance with the stimulation parameters programmed by the external control device, electrical pulses can be delivered from the neurostimulator to the stimulation electrode(s) to stimulate or activate a volume of tissue in accordance with a set of stimulation parameters and provide the desired efficacious therapy to the patient. The best stimulus parameter set will typically be one that delivers appropriate stimulation energy to the volume of tissue that is targeted for therapeutic benefit (e.g., treatment of pain), while minimizing the volume of non-target tissue that is stimulated.

In certain scenarios, it may be desirable to track the physical activity (e.g., activity level or body manipulations) of the patient that has received the implantable neurostimulation system, which provides an indication of the efficacy of the therapy provided by the stimulation system; that is, the more efficacious the therapy, the more diurnally active the patient will be. Thus, knowledge of the physical activity of the patient over a period of time in which therapeutic stimulation is applied to the patient may be used by a physician or clinician to prescribe drugs, reprogram or upgrade the IPG, or implement or modify other therapeutic regimens (such as physical or occupational therapy). Knowledge of the physical activity of the patient may also be used to adapt the therapy provided by the stimulation system in real time, so that the stimulation is consistently provided to the patient at an efficacious and/or comfortable level.

In other scenarios, it may be desirable to detect a posture or postural change (e.g., standing up, lying down, trunk twisting, bending, etc.). This is because the stimulation leads will tend to migrate relative to themselves, as well as relative to the tissue (e.g., in the case of SCS, a stimulation lead may move within the epidural space in which it is implanted) to be stimulated as the patient undergoes postural changes. With knowledge of the posture or the occurrence of a postural change, the therapy may be adjusted, so that the stimulation is consistently provided to the patient at an efficacious and/or comfortable level.

There is a need to provide an efficient and effective sensing device that indicates patient activity, a posture, or postural change of a patient implanted with a medical device.

### SUMMARY OF THE INVENTION

In accordance with a first aspect of the present inventions, an implantable medical device is provided. The implantable medical device comprises a medical component configured for performing a medical function (e.g., a therapeutic function, such as neurostimulation) in a patient. The implantable medical device further comprises an orientation sensitive component including a housing having a cavity, a movable object configured for being displaced within the cavity in response to the change in the direction of a force applied to the movable object, and a plurality of fixed sensors spaced apart within the cavity for sensing a location of the movable object within the cavity. The implantable medical device further comprises monitoring circuitry configured for determining the orientation of the implantable medical device based on the sensed location of the movable object. In an optional embodiment, the implantable medical device further comprises a casing containing the operative element, the orientation sensitive component, and the monitoring circuitry. In another optional embodiment, the implantable medical device further comprises telemetry circuitry configured for transmitting information indicating the determined orientation of the implantable medical device to an external device.

The fixed sensors may respectively correspond to different orientations, with each of the fixed sensors being configured for sensing when the movable object is adjacent the respective fixed sensor. In this case, the monitoring circuitry may be configured for identifying the orientation corresponding to the fixed sensor to which the movable object is adjacent as the determined orientation of the implantable medical device. The cavity and the movable object maybe geometrically similar. For example, the cavity and the movable object may be spherical.

In one embodiment, the plurality of fixed sensors may comprise a pair of sensors respectively affixed to opposing sides of the cavity, such that the monitoring circuitry is configured for bilaterally determining the orientation of the implantable medical device in at least one spatial dimension. In another embodiment, the plurality of fixed sensors may comprise a plurality of orthogonal pairs of sensors, with each pair of sensors being respectively affixed to opposing sides of the cavity, such that the monitoring circuitry is configured for bilaterally determining the orientation of the implantable medical device in at least two spatial dimensions. In still another embodiment, the plurality of fixed sensors comprises three orthogonal pairs of sensors, with each pair of sensors being respectively affixed to opposing sides of the cavity, such that the monitoring circuitry is configured for bilaterally determining the orientation of the implantable medical device in three spatial dimensions.

In one embodiment, each of the fixed sensors comprises a pair of electrical contacts, the movable object is a movable electrical contact configured for being placed into mechanical contact with each of the pair of electrical contacts one at a time to electrically couple each pair of electrical contacts to each other. In this case, the monitoring circuitry may be configured for sensing the electrical coupling between the each pair of electrical contacts. The monitoring circuitry may include an electrical energy source coupled to reference voltage via a plurality of circuit branches, the plurality of circuit branches may respectively include the plurality of pairs of electrical contacts, and the movable object may be configured for changing the electrical characteristics of each of the plurality of circuit branches when placed into mechanical contact with the each pair of electrical contacts. In this case, the monitoring circuitry may be configured for sensing the electrical characteristic change in the each circuit branch to sense the electrical coupling between each pair of electrical contacts. The monitoring circuitry may include a detector and a multiplexer. The multiplexer may have inputs respectively coupled to the circuit branches, and an output coupled to the detector, such that the detector can sequentially sense the electrical characteristic changes of the circuit branches.

In accordance with a second aspect of the present inventions, an orientation sensitive component for an implantable medical device is provided. The orientation sensitive component comprises a housing having a cavity, a movable object configured for being displaced within the cavity in response to the change in the direction of a force applied to the movable object, and a plurality of fixed sensors spaced apart within the cavity for sensing a location of the movable object within the cavity.

The fixed sensors may respectively correspond to different orientations, and each of the fixed sensors may be configured for sensing when the movable object is adjacent the respective fixed sensor. The cavity and the movable object maybe geometrically similar. For example, the cavity and the movable object may be spherical. In one embodiment, the plurality of fixed sensors may comprise a pair of sensors respectively affixed to opposing sides of the cavity. In another embodiment, the plurality of fixed sensors may comprise a plurality of orthogonal pairs of sensors, with each pair of sensors being respectively affixed to opposing sides of the cavity. In still another embodiment, the plurality of fixed sensors comprises three orthogonal pairs of sensors, with each pair of sensors being respectively affixed to opposing sides of the cavity. Each of the fixed sensors may comprise a pair of electrical contacts, and the movable object may be a movable electrical contact configured for being placed into mechanical contact with each of the pair of electrical contacts one at a time to electrically couple each pair of electrical contacts to each other.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of a Spinal cord Stimulation (SCS) system constructed in accordance with one embodiment of the present inventions;

FIG. 2 is a perspective view of the arrangement of the SCS system of FIG. 1 with respect to a patient;

FIG. 3 is a profile view of an implantable pulse generator (IPG) and percutaneous leads used in the SCS system of FIG. 1;

FIG. 4 is a block diagram of the internal components of the IPG of FIG. 3;

FIGS. 5a-5e are plan views of an orientation sensitive component in various states that can be used in the IPG of FIG. 3; and

FIGS. 6a and 6b are circuit diagrams of monitoring circuitry that can be used in the IPG of FIG. 3 to determine the orientation of the IPG based on signals received from the orientation sensitive component of FIGS. 5a-5e.

### DETAILED DESCRIPTION OF THE EMBODIMENTS

The description that follows relates to a spinal cord stimulation (SCS) system. However, it is to be understood that while the invention lends itself well to applications in SCS, the invention, in its broadest aspects, may not be so limited. Rather, the invention may be used with any type of implantable electrical circuitry used to stimulate tissue. For example, the present invention may be used as part of a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neurostimulator configured to treat urinary incontinence, sleep apnea, shoulder sublaxation, headache, etc. Furthermore, the present invention may be used with other non-electrical-based implantable stimulation devices, such as implantable drug pumps, and even with non-therapeutic implantable devices, such as devices used to monitor, record, and store recordings of physiological information within the patient.

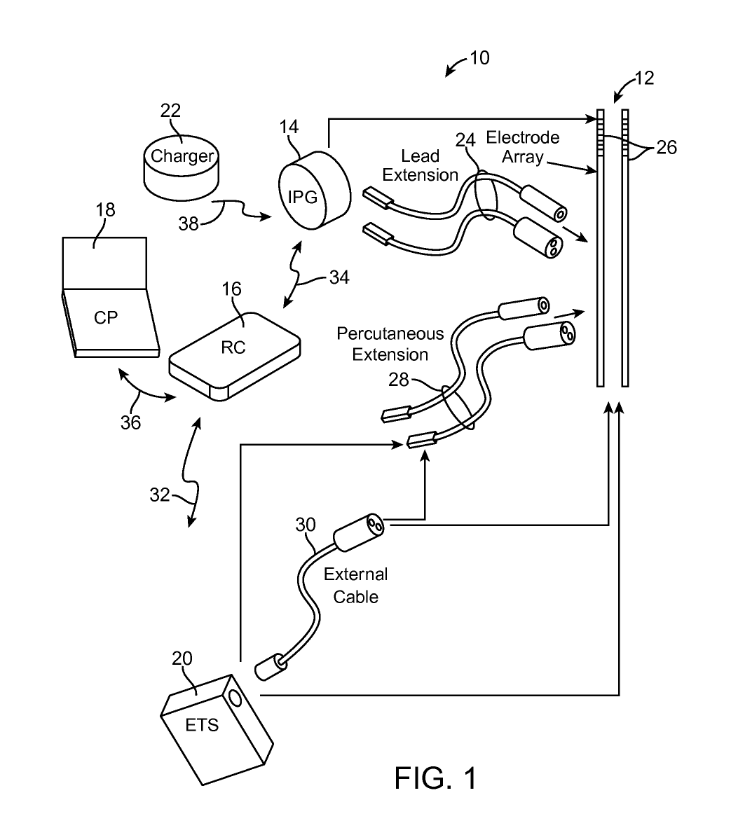

Turning first to FIG. 1, an exemplary SCS system 10 generally includes a plurality (in this case, two) of implantable neurostimulation leads 12, an implantable pulse generator (IPG) 14, an external remote controller RC 16, a clinician's programmer (CP) 18, an external trial stimulator (ETS) 20, and an external charger 22.

The IPG 14 is physically connected via one or more percutaneous lead extensions 24 to the neurostimulation leads 12, which carry a plurality of electrodes 26 arranged in an array. In the illustrated embodiment, the neurostimulation leads 12 are percutaneous leads, and to this end, the electrodes 26 are arranged in-line along the neurostimulation leads 12. As will be described in further detail below, the IPG 14 includes a medical component in the form of pulse generation circuitry, which delivers electrical stimulation energy in the form of a pulsed electrical waveform (i.e., a temporal series of electrical pulses) to the electrode array 26 in accordance with a set of stimulation parameters. In alternative embodiments, the medical component may be something other than pulse generation circuitry, e.g., a drug pump or a monitoring device for recording physiological data.

The ETS 20 may also be physically connected via the percutaneous lead extensions 28 and external cable 30 to the neurostimulation leads 12. The ETS 20, which has similar pulse generation circuitry as the IPG 14, also delivers electrical stimulation energy in the form of a pulse electrical waveform to the electrode array 26 accordance with a set of stimulation parameters. The major difference between the ETS 20 and the IPG 14 is that the ETS 20 is a non-implantable device that is used on a trial basis after the neurostimulation leads 12 have been implanted and prior to implantation of the IPG 14, to test the responsiveness of the stimulation that is to be provided. Thus, any functions described herein with respect to the IPG 14 can likewise be performed with respect to the ETS 20. Further details of an exemplary ETS are described in U.S. Pat. No. 6,895,280, which is expressly incorporated herein by reference.

The RC 16 may be used to telemetrically control the ETS 20 via a bi-directional RF communications link 32. Once the IPG 14 and neurostimulation leads 12 are implanted, the RC 16 may be used to telemetrically control the IPG 14 via a bi-directional RF communications link 34. Such control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. As will be described in further detail below, the CP 18 provides clinician detailed stimulation parameters for programming the IPG 14 and ETS 20 in the operating room and in follow-up sessions.

The CP 18 may perform this function by indirectly communicating with the IPG 14 or ETS 20, through the RC 16, via an IR communications link 36. Alternatively, the CP 18 may directly communicate with the IPG 14 or ETS 20 via an RF communications link (not shown). The clinician detailed stimulation parameters provided by the CP 18 are also used to program the RC 16, so that the stimulation parameters can be subsequently modified by operation of the RC 16 in a stand-alone mode (i.e., without the assistance of the CP 18).

The external charger 22 is a portable device used to transcutaneously charge the IPG 14 via an inductive link 38. Once the IPG 14 has been programmed, and its power source has been charged by the external charger 22 or otherwise replenished, the IPG 14 may function as programmed without the RC 16 or CP 18 being present.

For purposes of brevity, the details of the RC 16, CP 18, ETS 20, and external charger 22 will not be described herein. Details of exemplary embodiments of these devices are disclosed in U.S. Pat. No. 6,895,280, which is expressly incorporated herein by reference.

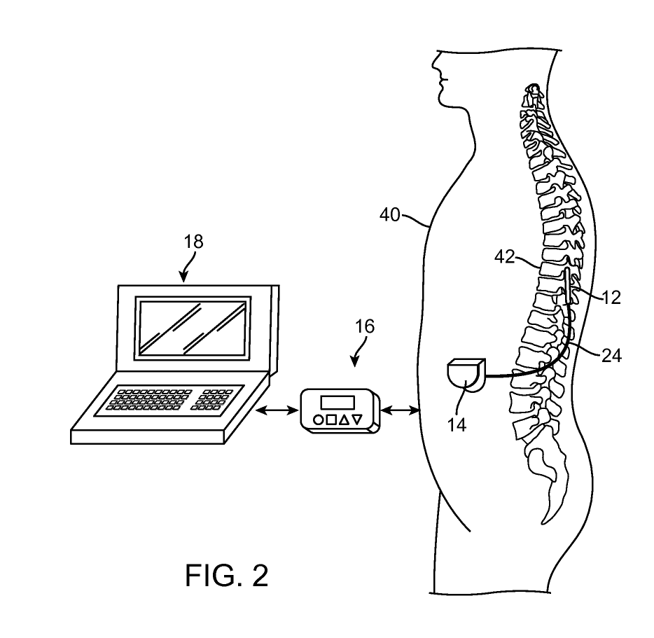

As shown in FIG. 2, the neurostimulation leads 12 are implanted within the spinal column 42 of a patient 40. The preferred placement of the neurostimulation leads 12 is adjacent, i.e., resting upon, the spinal cord area to be stimulated. Due to the lack of space near the location where the neurostimulation leads 12 exit the spinal column 42, the IPG 14 is generally implanted in a surgically-made pocket either in the abdomen or above the buttocks. The IPG 14 may, of course, also be implanted in other locations of the patient's body. The lead extensions 24 facilitate locating the IPG 14 away from the exit point of the neurostimulation leads 12. As there shown, the CP 18 communicates with the IPG 14 via the RC 16.

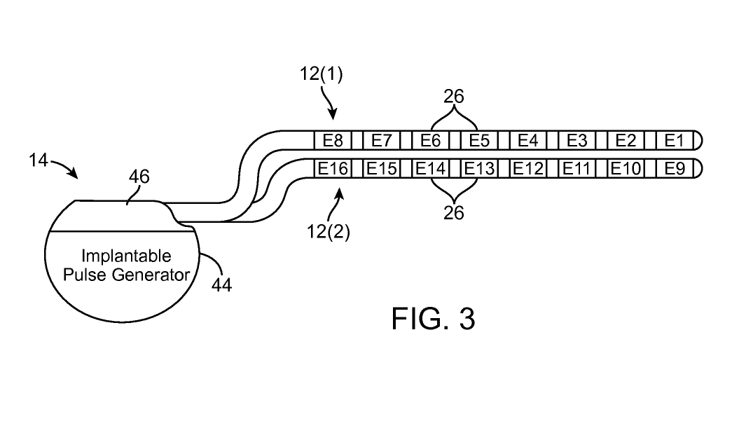

Referring now to FIG. 3, the features of the neurostimulation leads 12 and the IPG 14 will be briefly described. One of the neurostimulation leads 12(1) has eight electrodes 26 (labeled E1-E8), and the other neurostimulation lead 12(2) has eight electrodes 26 (labeled E9-E16). The actual number and shape of leads and electrodes will, of course, vary according to the intended application. The IPG 14 comprises an outer case 44 for housing the electronic and other components (described in further detail below), and a connector 46 to which the proximal ends of the neurostimulation leads 12 mates in a manner that electrically couples the electrodes 26 to the electronics within the outer case 44. The outer case 44 is composed of an electrically conductive, biocompatible material, such as titanium, and forms a hermetically sealed compartment wherein the internal electronics are protected from the body tissue and fluids. In some cases, the outer case 44 may serve as an electrode.

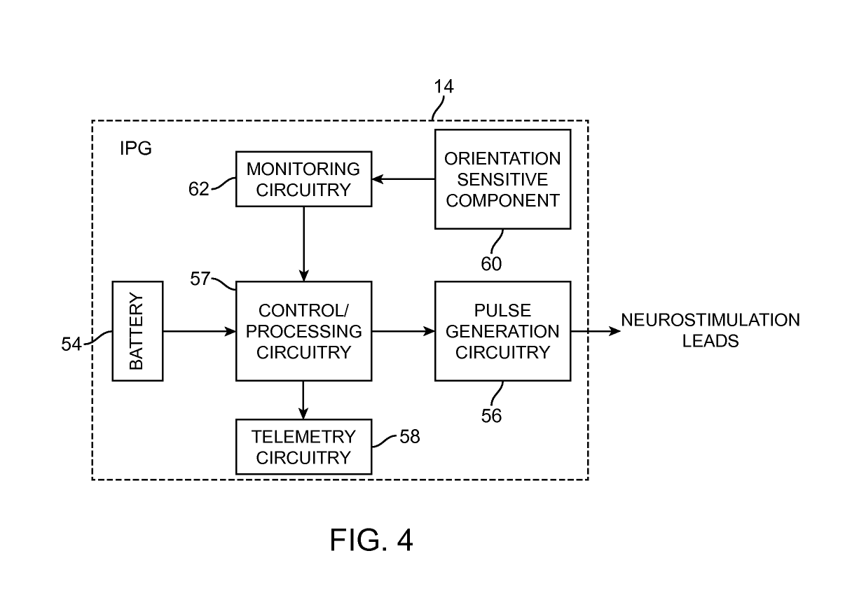

Referring further to FIG. 4, includes a battery 54 for providing power to the IPG 14, pulse generation circuitry 56 that delivers the electrical stimulation energy in the form of a pulsed electrical waveform to the electrode array 26 in accordance with a set of stimulation parameters programmed into the IPG 14, control/processing circuitry 57 for controlling the operation of the IPG 14 in accordance with a selected operating program and stimulation parameters, and telemetry circuitry 58 for transmitting control and status information between the IPG 14 and the RC 16/CP 18. Further details discussing the detailed structure and function of IPGs are described more fully in U.S. Pat. Nos. 6,516,227 and 6,993,384, which are expressly incorporated herein by reference.

It should be noted that rather than an IPG, the SCS system 10 may alternatively utilize an implantable receiver-stimulator (not shown) connected to the neurostimulation leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the stimulation in accordance with the control signals.

Significantly, the IPG 14 further includes an orientation sensitive component 60 configured for sensing an orientation of the IPG 14, and monitoring circuitry 62 configured for determining the orientation of the IPG 14 based on the sensed orientation, which orientation information can be transmitted to the RC 16 or CP 18 via the telemetry circuitry 58. The RC 16 or CP 18 may, in turn, reprogram the pulse generation circuitry 56 with new stimulation parameters in order to maintain optimal or otherwise effective stimulation. Alternatively, the control/processing circuitry 57 in the IPG 14 may automatically reprogram the stimulation parameters to maintain optimal or at least effective stimulation based on the determined orientation of the IPG 14.

In one example, the orientation information may indicate a posture or postural change of the patient in which the IPG 14 is implanted, and based on this posture or postural change, the pulse generation circuitry 56 may be reprogrammed to address the new posture or postural change. In one example, a look-up table containing different orientation information and corresponding stimulation parameter adjustments can be stored and subsequently accessed to effect the reprogramming of the pulse generation circuitry 56. In another example, the frequency of the changes in the orientation information may indicate a level of patient activity (e.g., the higher the frequency of changes, the higher the level of patient activity), which can be used to reprogram the pulse generation circuitry 56.

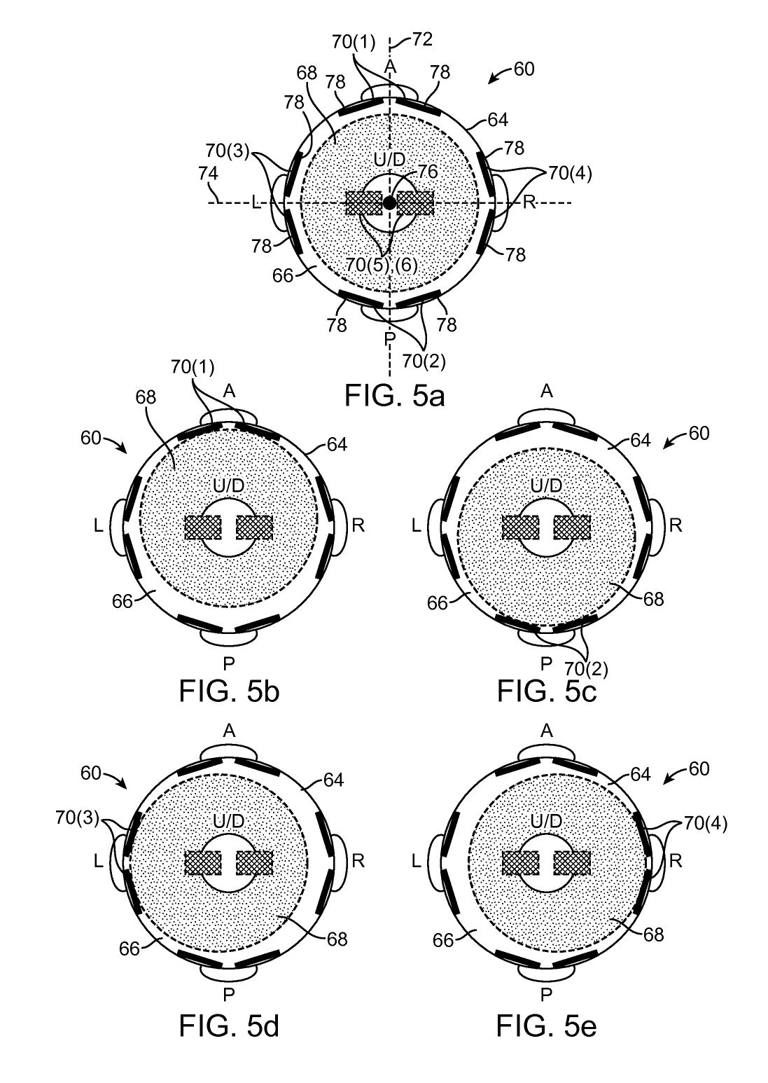

With further reference to FIGS. 5a-5e, the orientation sensitive component 60 generally comprises a housing 64 having a shaped cavity 66, a movable object 68 configured for being displaced within the cavity 66 in response to the change in the direction of a force applied to the movable object 68, and a plurality of fixed sensors 70 spaced apart within the cavity 66 for sensing a location of the movable object 68 within the cavity 66.

The housing 64 may be composed of a suitably rigid material, which preferably is at least electrically insulative on its inner surface where the sensors 70 are mounted. For example, the housing may be composed of polyetheretherketone (PEEK). It is preferred that the cavity 66 and the movable object 68 be geometrically similar, and that the cavity 66 be slightly larger than the movable object 68, so that displacement of the movable object 68 within the cavity 66 is limited to a predetermined number of positions, as will be described in further detail below. In the illustrated embodiment, the cavity 66 and the movable object 68 are both spherical in nature, such that the movable object 68 may roll around in the cavity 66. In alternative embodiments, the cavity 66 and the movable object 68 may have a different shape, e.g., cubic, cylindrical, pyramidal, etc. Ultimately, the shape of the cavity 66 and movable object 68 will depend on the number and direction of the orientations to be sensed. In alternative embodiments, geometrically similarity between the cavity 66 and the movable object 68 may not always be necessary. For example, a spherical movable object 68 within a cubic cavity 66 may work.

Significantly, the movable object 68 will move generally in accordance with the orientation of the IPG 14. In particular, a force (e.g., due to gravity or centripetal acceleration) applied to the movable object 68 in a specific direction will cause the movable object 68 to generally move in that direction relative to the housing 64. Thus, assuming that the IPG 14 is oriented relative to the applied force in a particular manner, such that a reference side of the IPG 14 faces in a particular direction, the movable object 68 will generally move relative to the housing 64 in that direction as allowed by the confines of the cavity 66.

In the illustrated embodiment, six sensors 70 are affixed to the inner surface of the housing 64 at six different positions around the cavity 66 using suitable means, such as bonding or welding. The six sensors 70 are arranged as three orthogonal pairs of sensors 70, with a first pair of sensors 70(1), 70(2) disposed along a first axis 72 on opposite sides of the cavity 66 at nominal anterior (A) and posterior (P) positions; a second pair of sensors 70(3), 70(4) disposed along a second axis 74 (orthogonal to the first axis 72) on opposite sides of the cavity 66 at nominal left (L) and right (R) positions; and a third pair of sensors 70(5) (second sensor not shown) disposed along a third axis 76 (perpendicular to the drawing sheet and orthogonal to the first and second axes 72, 74) on opposite sides of the cavity 66 at nominal up (U) and down (D) positions.

Each of the sensors 70 is configured for sensing when the movable object 70 is adjacent to it. For example, each of the sensors 70 may be activated or triggered only when the movable object 68 is in the position corresponding to the respective sensor 70. For example, none of the sensors 70 are activated when the movable object 68 is in a neutral position (FIG. 5a); the anterior sensor 70(1) is activated when the movable object 68 is in the anterior position (FIG. 5b); the posterior sensor 70(2) is activated when the movable object 68 is in the posterior position (FIG. 5c); the left sensor 70(3) is activated when the movable object 68 is in the left position (FIG. 5d); the right sensor 70(4) is activated when the movable object 68 is in the right position (FIG. 5e); the upper sensor 70(5) is activated when the movable object 68 is in the up position (not shown); and the down sensor is activated when the movable object 68 is in the down position (not shown). In the illustrated embodiment, the sensors 70 are spaced far enough apart, such that only one at a time can be activated by the movable object 68.

It can thus be appreciated that, assuming that the orientation sensitive component 60 is mounted within the IPG 14 in a predetermined and known manner, the activation of a specific sensor 70 will indicate the specific orientation of the IPG 14. Thus, each sensor 70 corresponds to a specific orientation of the IPG 14, and as such, the monitoring circuitry 62 can correlate the orientation corresponding to the activated sensor 70 to the orientation of the IPG 14. Notably, because each of the three sensor pairs 70 are oppositely disposed relative to each other, the monitoring circuitry 62 can bilaterally determine the orientation of the IPG 14 in three spatial dimensions.

In the illustrated embodiment, each of the sensors 70 comprises a pair of electrical contacts 78, and the movable object 68 takes the form of a movable electrical contact that is capable of being placed into mechanical contact with the pairs of electrical contacts 78 one at a time to electrically couple the respective pair of electrical contacts 78 to each other. The electrical contacts 78 of each pair are separated from each other a suitable distance that allows the movable object 68 to be placed into mechanical contact with both contacts 78. Thus, it can be appreciated that each electrical contact pair 78 acts as a switch that can be placed between an open state (movable object 68 not in contact with electrical contact pair 78) and a closed state (movable object in contact with electrical contact pair 78).

The monitoring circuitry 62 is configured for sensing the electrical coupling between each pair of electrical contacts 78; i.e., the open/close status of the electrical contact pair. In alternative embodiments, the sensors may take the form of other sensors that are capable of sensing the location of the movable object 68, e.g., a source-detector configuration, such as light sources and optical sensors, in which case, the monitoring circuitry 62 may monitor changes in the incident light due to movement of an optically opaque or reflective movable object. Other sensors may include, e.g., pressure sensors, piezoelectric sensors, optical-electrical sensors, capacitive sensors, etc. In these case, these sensors may not operate as a switch, but rather operate to change the resistance and/or capacitive of an electric circuit.

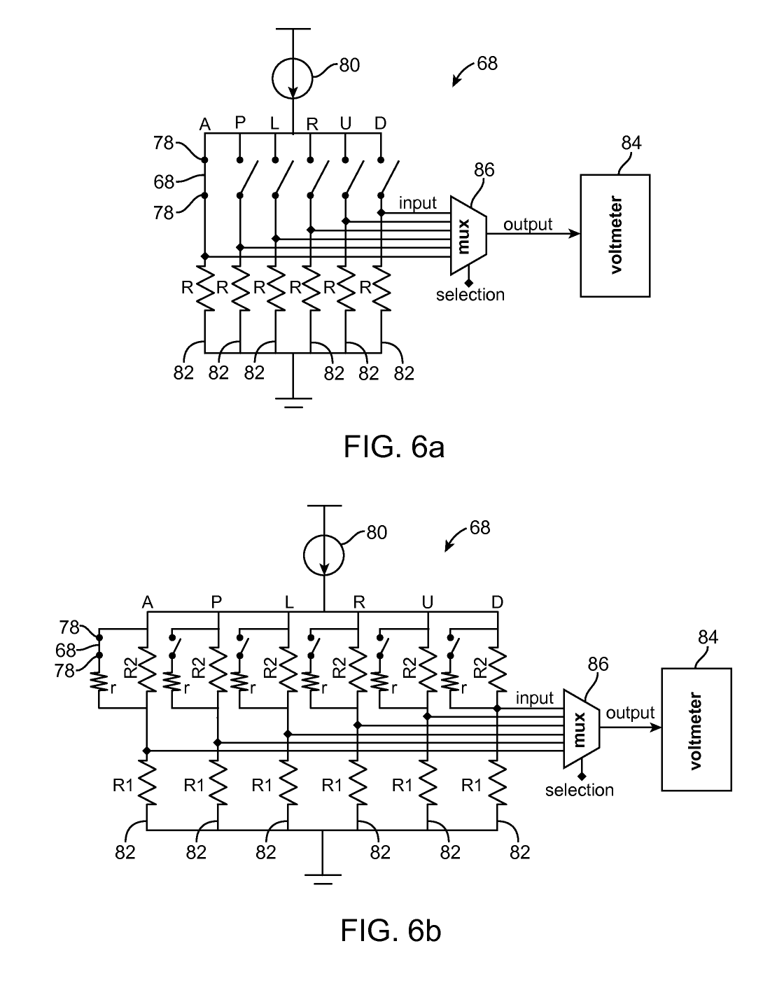

In the case where the sensors 70 take the form of electrical contact pairs 78, the monitoring circuitry 62, in the embodiments illustrated in FIGS. 6a and 6b, includes an electrical energy source 80 (in these cases, a current source) coupled to ground (or alternatively a reference voltage other than zero) via a plurality of identical circuit branches 82 (designated A, P, L, R, U, and D), which respectively include the plurality of electrode contact pairs 78. In the illustrated embodiment, the electrical energy source 80 takes the form of a current source, although other types of electrical energy sources, such as a voltage source, can be used. When the movable object 68 is placed into mechanical contact with one of the electrode contact pairs 78, the electrical characteristics of the respective circuit branch 82 changes. Thus, the monitoring circuitry 62 is capable of monitoring the connection status of each of the electrode contact pairs 78, and thus, the orientation of the IPG 14.

In the illustrated embodiment, the monitoring circuitry 62 includes a voltmeter 84 and a multiplexer 88 that can be operated via selection signals generated by control circuitry (not shown), such that the voltmeter 84 can sequentially measure the change in the electrical characteristics in the form of a voltage change within each of the circuit branches 82. That is, the inputs of the multiplexer 86 are respectively coupled to the circuit branches 82, such that the voltages on the circuit branches 82 can be selectively coupled to the output of the multiplexer 86 for sequential measurement by the voltmeter 84.

In the embodiment illustrated in FIG. 6a, the circuit branches 82 are resistive and include equally valued resistances R coupled between the electrode contact pairs 78 and ground, such that a voltage can be measured across the resistor R of a selected resistive branch 82 when the corresponding electrode contact pair 78 is shorted (switch closed), and a ground or reference voltage can be measured across the resistor R of a selected resistive branch 82 when the corresponding electrode contact pair 78 is open (switch open). In this case, the orientation corresponding to the resistive branch 82 on which an actual voltage (as opposed to no voltage) is measured will be the determined IPG orientation.

In the embodiment illustrated in FIG. 6b, the resistive branches 82 include equally valued resistances R1 coupled between the electrode contact pairs 78 and ground, and equally valued resistances R2 coupled in parallel with the respective electrode contact pairs 78, such that a first voltage can be measured across the resistor R1 of a selected resistive branch 82 when the corresponding electrode contact pair 78 is coupled to each other via an additional resistance r provided by the movable object 68, and a second different voltage can be measured across the resistor R1 of a selected resistive branch 82 when the corresponding electrode contact pair 78 is not coupled by the movable object 68. In this case, the orientation corresponding to the resistive branch 82 having a substantially different voltage than the voltages on the other resistive branches 82 will be the determined IPG orientation.

Although the monitoring circuitry 62 in the illustrated embodiments measures voltage, it should be appreciated that other electrical parameters, such as impedance/resistance, current, etc, can be measured to identify changes in the electrical characteristics of the circuit branches.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. An implantable medical device, comprising:
a medical component configured for performing a medical function in a patient;
an orientation sensitive component including a housing having a cavity, a movable object configured for being displaced within the cavity in response to the change in the direction of a force applied to the movable object, and a plurality of fixed sensors spaced apart within the cavity for sensing a location of the movable object within the cavity; and
monitoring circuitry configured for determining the orientation of the implantable medical device based on the sensed location of the movable object.

2. The implantable medical device of claim 1, wherein the plurality of fixed sensors respectively correspond to a plurality of different orientations, each of the fixed sensors is configured for sensing when the movable object is adjacent the respective fixed sensor, and the monitoring circuitry is configured for identifying the orientation corresponding to the fixed sensor to which the movable object is adjacent as the determined orientation of the implantable medical device.

3. The implantable medical device of claim 1, wherein the cavity and the movable object are geometrically similar.

4. The implantable medical device of claim 3, wherein the cavity and the movable object are spherical.

5. The implantable medical device of claim 1, wherein the plurality of fixed sensors comprises a pair of sensors respectively affixed to opposing sides of the cavity, such that the monitoring circuitry is configured for bilaterally determining the orientation of the implantable medical device in at least one spatial dimension.

6. The implantable medical device of claim 1, wherein the plurality of fixed sensors comprises a plurality of orthogonal pairs of sensors, each pair of sensors being respectively affixed to opposing sides of the cavity, such that the monitoring circuitry is configured for bilaterally determining the orientation of the implantable medical device in at least two spatial dimensions.

7. The implantable medical device of claim 1, wherein the plurality of fixed sensors comprises three orthogonal pairs of sensors, each pair of sensors being respectively affixed to opposing sides of the cavity, such that the monitoring circuitry is configured for bilaterally determining the orientation of the implantable medical device in three spatial dimensions.

8. The implantable medical device of claim 1, wherein each of the fixed sensors comprises a pair of electrical contacts, the movable object is a movable electrical contact configured for being placed into mechanical contact with each of the pair of electrical contacts one at a time to electrically couple the each pair of electrical contacts to each other, and the monitoring circuitry is configured for sensing the electrical coupling between the each pair of electrical contacts.

9. The implantable medical device of claim 8, wherein the monitoring circuitry includes an electrical energy source coupled to a reference voltage via a plurality of circuit branches, wherein the plurality of circuit branches respectively include the plurality of pairs of electrical contacts, wherein the movable object is configured for changing the electrical characteristics of each of the plurality of circuit branches when placed into mechanical contact with the each pair of electrical contacts, and the monitoring circuitry is configured for sensing the electrical characteristic change in the each circuit branch to sense the electrical coupling between the each pair of electrical contacts.

10. The implantable medical device of claim 9, wherein the monitoring circuitry includes a detector and a multiplexer, the multiplexer having inputs respectively coupled to the circuit branches, and an output coupled to the detector, such that the detector can sequentially sense the electrical characteristic changes of the circuit branches.

11. The implantable medical device of claim 1, further comprising a casing containing the operative element, the orientation sensitive component, and the monitoring circuitry.

12. The implantable medical device of claim 1, further comprising telemetry circuitry configured for transmitting information indicating the determined orientation of the implantable medical device to an external device.

13. An orientation sensitive component for an implantable medical device, comprising:
a housing having a cavity;
a movable object configured for being displaced within the cavity in response to the change in the direction of a force applied to the movable object; and
a plurality of fixed sensors spaced apart within the cavity for sensing a location of the movable object within the cavity.

14. The orientation sensitive component of claim 13, wherein the plurality of fixed sensors respectively correspond to a plurality of different orientations, and each of the fixed sensors is configured for sensing when the movable object is adjacent the respective fixed sensor.

15. The orientation sensitive component of claim 13, wherein the cavity and the movable object are geometrically similar.

16. The orientation sensitive component of claim 15, wherein the cavity and the movable object are spherical.

17. The orientation sensitive component of claim 13, wherein the plurality of fixed sensors comprises a pair of sensors respectively affixed to opposing sides of the cavity.

18. The orientation sensitive component of claim 13, wherein the plurality of fixed sensors comprises a plurality of orthogonal pairs of sensors, each pair of sensors being respectively affixed to opposing sides of the cavity.

19. The orientation sensitive component of claim 13, wherein the plurality of fixed sensors comprises three orthogonal pairs of sensors, each pair of sensors being respectively affixed to opposing sides of the cavity.

20. The orientation sensitive component of claim 13, wherein each of the fixed sensors comprises a pair of electrical contacts, the movable object is a movable electrical contact configured for being placed into mechanical contact with each of the pair of electrical contacts one at a time to electrically couple the each pair of electrical contacts to each other.
